(19) United States
(12) Patent Application Publication
(10) Pub. No.: US 2009/0104655 A1
(43) Pub. Date: Apr. 23, 2009

(54) Fermentation Method and Apparatus for its Implementation

(75) Inventors: Jochen Buchs, Aachen (DE); Doris Klee, Aachen (DE); Barbara Dittrich, Aachen (DE); Markus Jeude, Alsdorf (DE)

Correspondence Address: COHEN, PONTANI, LIEBERMAN & PAVANE LLP, 551 FIFTH AVENUE, SUITE 1210, NEW YORK, NY, 10176, US

(73) Assignee: RWTH AACHEN, Aachen (DE)

(21) Appl. No.: 11/920,108

(22) PCT Filed: Apr. 26, 2006
(86) PCT No.: PCT/EP2006/003838

§ 371 Date: Nov. 8, 2007

(30) Foreign Application Priority Data
May 9, 2005 (DE) 10 2005 022 045.2

(51) Int. Cl. C12P 1/00; C12M 1/22

(52) U.S. Cl. 435/41; 435/305.2

(57) ABSTRACT

In order to develop and optimize metering strategies for fed-batch fermentations in two or more reaction vessels, release systems containing nutrients are attached to the inner surfaces of the reaction vessels in areas that will come into contact with culture liquid placed in the vessels.

The invention pertains to a fermentation method with controlled feed of nutrients into the culture liquid of several reaction vessels, where the nutrients from at least one release system are released into each reaction vessel. The invention also pertains to an apparatus for implementing a fermentation process with controlled feed of nutrients from release systems into the culture liquid of several reaction vessels.

For production processes in the field of bioengineering, the method based on supplying nutrients, the so-called “fed-batch” method, has proven to be especially advantageous. Through the supply of nutrients during the fermentation, it is possible to achieve dramatic increases in the growth and production phases in comparison with the pure batch method. The growth of the microorganisms depends strongly on the composition of the culture liquid, especially on the composition of the nutrients present in the culture liquid. If, for example, growth is inhibited by an excess of nutrients, by insufficient water activity, or by excessive osmotic pressure, catabolite repression will occur, or if, during the fermentation process, an overflow mechanism goes into action, during which, for example, lactate, acetate, or ethanol is separated, the optimal microorganisms and culture conditions cannot be found with batch fermentation.

Fed-batch fermentation in a conventional bioreactor differs from batch fermentation in that the reaction vessel is only partially filled at the start of the fermentation. Only during the course of the fermentation process does the volume in the reaction vessel reach the maximum value through the controlled supply of nutrient solutions. The time at which the controlled feed of nutrient solutions begins depends on the fermentation method being used.

One of the goals of fed-batch fermentation in many cases is to keep, for a certain period of time, the concentration of limiting nutrients or the concentration of a precursor in the reaction vessel in a low concentration range found to be advantageous for the biological reaction. Another goal of fed-batch fermentation can be to allow the microorganisms merely to grow during the first phase and then, after a certain time, to initiate the conversion to the desired product by starting to feed the nutrients in a controlled manner.

In most cases, the nutrient feed for a production process by the fed-batch method is developed empirically. The necessary experiments take a great a deal of time and equipment, and they must be carried out sequentially in agitated laboratory reactors. The standard instrument for developing fermentation production methods in the field of bioengineering, however, is the shake-flask technique. This approach makes it possible to study the influence of many important process parameters such as nutrient spectra, pH value, temperature, and trace element concentrations in parallel series of experiments.

So that the shake-flask technique can also be used to develop fed-batch processes, the fed-batch shake-flask technique was developed (Chemie Ingenieur Technik (68) 11/96). For this purpose, the simple shake-flask technique is combined with a precise metering technique adapted to small reaction volumes. The metering technique consists of a highly precise piston pump, which distributes the various nutrients over several shake flasks by means of a multi-way valve and the associated metering lines. A process computer controls the metering of the nutrients. As a result, it is possible to give each one of the shake flasks being used in parallel an individual metering profile on the basis of a metered amount-versus-time diagram. With this fed-batch shake-flask system, up to 14 flasks can be supplied in parallel with 4 different nutrients.

Although the known fed-batch shake-flask system makes it possible to achieve automated, parallel development and optimization of metering strategies for fed-batch fermentations on a laboratory scale, the number of reaction vessels which can be used in parallel is still limited because of the large amount of apparatus required. It is therefore almost impossible economically to screen a large number of different microorganisms (e.g., several hundred) to find those which deliver the optimal yield of microorganisms and/or product.

In the article entitled “Use of controlled-release polymer to feed ammonium to Streptomyces clavuligerus cephalosporin fermentations in shake flasks”, Appl. Microbiol. Biotechnol. (1985) 22:424-427), Lübbe et al. describe the encapsulation of the nutrients in a polymer matrix serving as a release system, which is added to a culture liquid in a shake flask. The release systems are in the form of polymer disks with a nutrient content of approximately 33% and a diameter of approximately 9 mm. The disks are suspended in the culture liquid, which comes in contact with all sides of the disk. Each shake flask contains two disk-like release systems, which slowly release the nutrients. The disadvantage is that the release systems can float on the swirling culture liquid in the shake flask. As a result, there is no longer any defined contact surface between the release system and the culture liquid, and the release rate is undetermined. In addition, the gas exchange at the surface of the culture liquid is impeded in an undefined manner.

Microtiter plates of plastic with, for example, 24, 48, or 96 wells have been known for several decades and are also used for the cultivation of microorganisms.

Proceeding on the basis of the prior art described above, the invention has the goal of developing a method of the type indicated above which allows screening under fed-batch conditions and makes possible the development and optimization of metering strategies for fed-batch fermentations with modest apparatus, where defined release rates can be achieved and the gas exchange at the surface of the culture liquid is not impeded.

An apparatus suitable for implementing the method is also proposed.

The goal is achieved in a method of the type indicated above in that the release systems are attached to the inside surfaces of the reaction vessels in an area which subsequently makes contact with the culture liquid.

The release systems are immobilized either on the bottoms or walls or on both the bottoms and the walls of the reaction vessels. In contrast to the prior art, the release system is not suspended in the culture liquid. The surface making contact with the culture liquid is therefore always the same. As a result, the nutrients held in the release system are released with defined kinetics. In addition, the gas exchange with the gas phase is not impeded.

A release system according to the invention guarantees that the nutrient to be released into the culture liquid will be present in a specific concentration over a period ranging in particular from a minimum of 1 hour to a maximum of 3 weeks.

In one embodiment of the inventive method, the nutrients are released at a constant mass flow rate adapted to the fermentation process. A constant feed can lead to a desirable limitation on the biological growth of the microorganisms.

The nutrients can also be supplied by the release system at a variable mass flow rate. If the mass flow rate increases exponentially, for example, it is possible to achieve a nearly constant nutrient concentration.

In another embodiment of the inventive method, the nutrients are supplied after a certain delay from the start of the fermentation in order to adapt the nutrient supply to a possible lag phase of the microorganisms. Once the nutrient feed begins after the delay, the mass flow rate can again be either variable or constant.

When the nutrient is the source of carbon, the release rate of the release systems is in the range of 0.1-10 gₙᵤₜᵣᵢₑₙₜ×cultivation day/(liter_{culture volume}×hour), preferably in the range of 1-2 gₙᵤₜᵣᵢₑₙₜ×cultivation day/(liter_{culture volume}×hour). If the limiting nutrient is a different component, the nutrient will be released at a slower rate in correspondence with the demand of the microorganisms.

The energy input into the culture liquid can be supplied by shaking the reaction vessels, for example, or by stirring the culture liquid. The stirring is preferably accomplished by magnetic stirrers. Another variant is a bubble column, in which air is blown under pressure into the lower part of the reactors.

An advantageous apparatus for implementing a fermentation with controlled feed of nutrients from release system into the culture liquid of several reaction vessels, especially according to one or more of Claims 1-7, is characterized in that
the reaction vessels are connected to each other, the connected reaction vessels being designed in particular to form a microtiter plate; in that
at least one release system is located in each reaction vessel; and in that
each release system is attached to an inside surface of one of the reaction vessels in an area which comes in contact with the culture liquid.

The inventive attachment of the release systems ensures defined release rates and the undisturbed gas exchange of the culture liquids. The attachment of the release systems can be accomplished by polymerizing the desired monomer to produce a polymer matrix in the reaction vessel. It is also possible to fabricate a film or disk to serve as the release system first, outside the reaction vessels. This film or disk is then divided into suitable pieces and glued or clamped to the reaction vessels.

The connection of the reaction vessels into a structural unit, especially into a microtiter plate known in and of itself, allows the streamlined performance of a large number of parallel fermentations in a very small space. Another advantage of connecting the reaction vessels to each other is that the unit, especially the microtiter plate, can be handled by laboratory robots, and thus the entire fermentation method can be automated.

Microtiter plates with integrated release systems attached to the inside surfaces, which have release kinetics adapted to the fed-batch cultivation of microorganisms, should preferably be designed for one-time use and packaged in sterile packaging.

The inventive apparatus, especially an apparatus making use of microtiter plates in sterile packaging for one-time use, significantly lowers the apparatus-related costs and reduces the amount of handling required in comparison with known fermentation methods with controlled feed of nutrients. The user simply removes the microtiter plate with its integrated release systems from the sterile packaging and is then able to use this plate immediately for the fed-batch cultivation of 96 different cultures, for example, with defined nutrient supply.

The use of microtiter plates with integrated release systems makes it easier to make available a defined concentration of specific nutrients in the test batches without the need for any special measuring or control equipment.

The release system can be formed out of a nutrient reservoir, which is encapsulated by a part of the inside surface of the well, especially the bottom of the well, and by a diffusion barrier, which is joined to the inside surface of the well. The polymer layer functioning as a diffusion barrier thus covers the nutrient reservoir.

Another solution consists in embedding the nutrients in the diffusion barrier itself, which then releases the nutrients in correspondence with their physical properties. A release system designed in this way has a height ranging from 50 μm to 4 mm, and preferably from 200 μm to 2 mm.

Finally, a release system formed by a nutrient reservoir (polymer matrix) embedded in a polymer can be considered for cases in which the nutrients are to be released in a time-delayed manner. The embedded nutrient reservoir is encapsulated by a part of the inside surface of the well and by a diffusion barrier, which is joined to the inside surface of the well. This diffusion barrier usually does not contain any nutrients, but it can contain small amounts of nutrients which act as pore-forming agents.

Nutrient crystals with a wide particle-size distribution can be incorporated into the polymer matrix. Narrow particle-size distributions are preferred, because as a result more well-defined release systems are obtained. The particle sizes of the nutrient crystals can range from 5 μm to 2 mm, but particle sizes from 50 μm to 500 μm are preferably used. In addition, nutrient particles with different defined particle-size distributions or mixtures of two more fractions, each with a narrow particle size distribution, can be used. The polymerization process used to form the polymer matrix can be controlled in such a way that the nutrients with different particle sizes are present on different levels in the release systems. Thus, for example, by defining the viscosity of the monomer liquid in advance and by taking advantage of the different settling rates of the differently sized particles during the polymerization process, the large nutrient particles can be induced to sink first, whereupon the smaller nutrient particles settle on top of them in the polymer matrix. When nutrient crystals with a single narrow particle-size distribution are used, furthermore, a stratified density distribution of the particles over the height of the polymer matrix can be obtained. As a result of this special preparation method, advantageous release kinetics can be obtained, which are specifically adapted to the needs of the microorganisms.

Natural and synthetic polymers can be used as the polymer materials for the release system. In particular, the following polymers and groups of polymers are suitable:

polysaccharides and their derivatives;

polysiloxanes;

polyacrylic acid and its derivatives;

polycarbonates;

polyolefins and their derivatives;

polycarboxylic acids and their derivatives;

polyethers and their derivatives;

polyesters and their derivatives;

polyamines and amides and their derivatives;

polysulfones and their derivatives;

polyurethanes;

polyvinyls and their derivatives, especially polyvinyl alcohols; and

copolymers of the polymers cited above and derivatives obtained by modification.

If, for example, alginate is used in combination with chitosan and polyamide as natural polymers, release times of over 6 hours are possible. With the synthetic polymer Eudragid, which was used experimentally as a layer surrounding a nutrient core, it was possible to achieve release times of more than 12 hours. Experiments with a diffusion barrier of silicone as a synthetic polymer were also conducted; nutrient crystals were suspended in it before crosslinking. The diffusion barrier with nutrients embedded in it in this way was able to provide a release time of approximately 3-250 hours.

In an advantageous embodiment of the invention, the diffusion barrier contains swelling bodies, which make the diffusion barrier permeable only after the swelling process has occurred. As a result, it is possible to delay the release of the nutrients during fermentations which include a so-called lag phase.

As pore-forming agents, soluble components in the diffusion barrier gradually form channels, which accelerate the release of the nutrients by increasing the mass flow rate. Through the selection of components with different solubilities, the release of the nutrients can be effectively controlled.

If the swelling bodies increase their volume as a function of the pH and/or temperature of the culture liquid and/or if the soluble components or pore-forming agents dissolve as a function of the pH and/or temperature of the culture liquid, the release of the nutrients can be controlled as a function of pH or temperature. For example, in the case of an acidifying culture liquid, the nutrients will not be released until the pH or the temperature reaches a certain value.

The invention is explained in greater detail below on the basis of the figures:

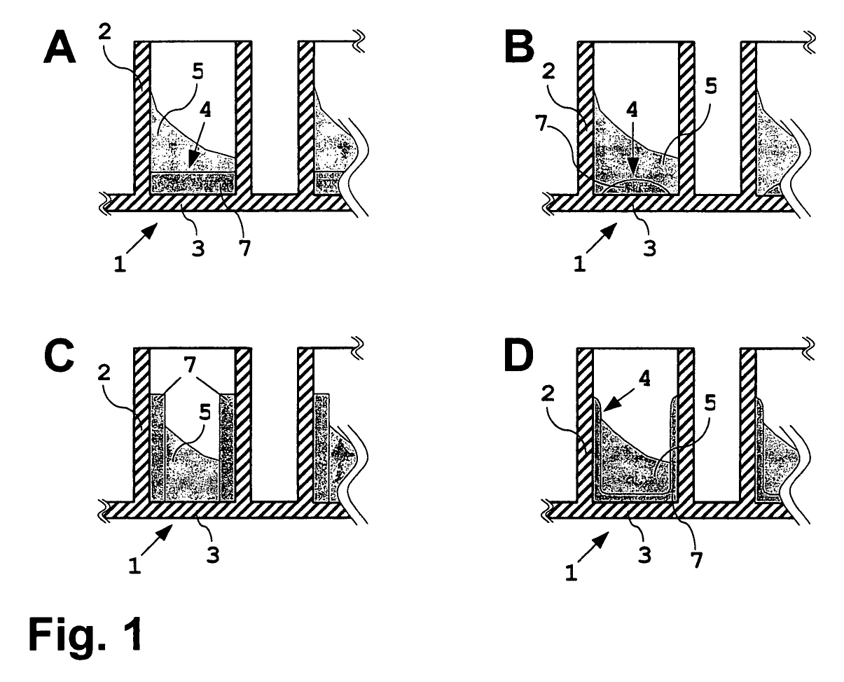

FIG. 1 shows schematic, cross-sectional side views of a reaction vessel of a microtiter plate for implementing the inventive method, where

FIG. 1A shows a reaction vessel with a release system on the bottom;

FIG. 1B shows another reaction vessel with a release system on the bottom;

FIG. 1C shows a reaction vessel with a hollow cylindrical release system on the side wall; and

FIG. 1D shows a reaction vessel with a release system which extends over the wall and the bottom of the vessel;

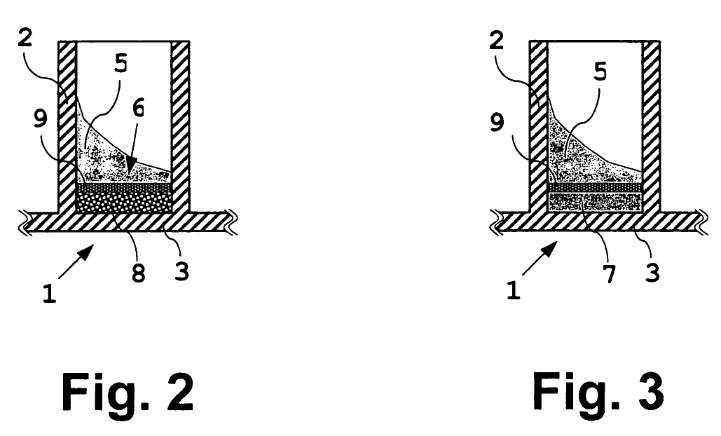

FIG. 2 shows a schematic, cross-sectional side view of a reaction vessel of a microtiter plate with a release system designed differently from that of FIG. 1; and

FIG. 3 shows a schematic, cross-sectional side view of a reaction vessel of a microtiter plate with a release system designed differently from that of FIGS. 1 and 2.

FIG. 1 shows a reaction vessel of a microtiter plate. Only part of the plate is shown. Overall, the plate contains a large number of these types of reaction vessels 1 in a uniform arrangement. The reaction vessels 1 of a microtiter plate are also called “wells”. They are connected permanently to each other and can therefore be easily handled in the course of automated processes. The individual reaction vessels 1 are hollow cylindrical bodies with closed bottoms. Each consists of a circumferential wall 2 and a bottom surface 3.

In the exemplary embodiment according to FIG. 1, the release system 4 is formed by a diffusion barrier 7 of polymer material, in which the nutrients are embedded (polymer matrix). The embedding of the nutrient particles is indicated in FIG. 1 by the white dots in the release systems 4.

According to FIG. 1A, the release system 4 extends over the entire bottom surface. The release system can be attached by polymerization of a monomer to form the polymer matrix in the reaction vessel.

Because the release systems are attached to the inside surfaces of the reaction vessels first and do not come in contact with the culture liquid 5 until a later time, the microtiter plates with the release systems attached to the inside surfaces can be prefabricated, placed in sterile packaging, and stored without difficulty until they are needed.

FIGS. 1B-1D show other possible ways in which the release systems can be attached. FIG. 1B shows a variant in which the release system 4 does not extend over the entire bottom surface 3; in the case of the variant according to FIG. 1C, a hollow cylindrical release system 4 is attached to the side wall 2 of the reaction vessel 1, whereas FIG. 1D shows a release system 4 extending across part of the side wall 2 and the bottom surface 3.

FIGS. 1A-1D also illustrate the point that the release systems 4 are all attached to the inside surfaces of the reaction vessels 1 in an area which subsequently comes in contact with the culture liquid 5, which is added at a later time.

The release system 6 according to FIG. 2 differs from the release system according to FIG. 1 in that the release system 6 is formed by a nutrient reservoir 8, which is enclosed by part of the inside surface of the well, namely, by the bottom 3 and the lower part of the side wall 2, and by a diffusion barrier 9, which is joined to the side wall 2 of the well. The polymer layer functioning as a diffusion barrier 9 thus covers the nutrient reservoir 8. The nutrients can therefore reach the culture 5 liquid only by passing through the diffusion barrier 9.

Finally, a release system according to FIG. 3 can be considered, especially in cases where the release of nutrients is to be delayed. This system is formed by a nutrient reservoir (polymer matrix) 7 embedded in a polymer, where the embedded nutrient reservoir (polymer matrix) 7 is encapsulated by part of the inside surface of the well and by a diffusion barrier 9, which is joined to the inside surface of the well. The diffusion barrier 9 does not contain any nutrients, as is also true for the diffusion barrier 9 shown in FIG. 2.

### LIST OF REFERENCE NUMBERS

| No. | Designation |
|---|---|
| 1 | reaction vessel |
| 2 | wall |
| 3 | bottom surface |
| 4 | release system |
| 5 | culture liquid |
| 6 | release system |
| 7 | diffusion barrier (polymer matrix with nutrient particles) |
| 8 | nutrient supply |
| 9 | diffusion barrier (polymer layer without nutrient particles) |

1.-17. (canceled)

18. A fermentation method with controlled feeding of nutrients into culture liquids of a plurality of reaction vessels, the method comprising:
providing a plurality of reaction vessels, each vessel having an inside surface comprising a bottom and a wall;
providing release systems containing nutrients;
attaching the release systems to the inside surfaces of the reaction vessels so that the release systems will come into contact with culture liquid placed in the vessels; and
placing a culture liquid into the reaction vessels so that fermentation can begin.

19. The fermentation method of claim 18 wherein the release systems are attached to at least one of the bottoms and the walls of the reaction vessels.

20. The fermentation method of claim 18 wherein the nutrients are released after a predetermined time delay from the beginning of fermentation.

21. The fermentation method of claim 18 wherein the nutrients are released by the release system into the culture liquid at a constant mass flow rate.

22. The fermentation method of claim 18 wherein the nutrients are released by the release system into the culture liquid at a variable mass flow rate.

23. The fermentation method of claim 18 further comprising shaking the reaction vessels during fermentation.

24. The fermentation method of claim 18 further comprising stirring the reaction vessels during fermentation.

25. A fermentation apparatus with controlled feeding of nutrients into culture liquids of a plurality of reaction vessels, the apparatus comprising:
a plurality of reaction vessels that are connected to each other, each vessel having an inside surface comprising a bottom and a wall; and
release systems attached to the inside surfaces of the reaction vessels so that the release systems will come into contact with culture liquid placed in the vessels, the release systems containing nutrients which can be released into culture liquid placed in the vessels.

26. The fermentation apparatus of claim 25 wherein the reaction vessels constitute wells of a microtiter plate.

27. The fermentation apparatus of claim 26 where the release systems are attached to at least one of the bottoms and the walls of the wells of the microtiter plate.

28. The fermentation apparatus of claim 25 wherein each said release system comprises a diffusion barrier which controls release of nutrients into the culture liquid.

29. The fermentation apparatus of claim 28 further comprising a nutrient reservoir containing the nutrients, the nutrient reservoir being encapsulated by the inside surface and the diffusion barrier.

30. The fermentation apparatus of claim 29 wherein the nutrients are also embedded in the diffusion barrier.

31. The fermentation apparatus of claim 28 wherein the nutrients are embedded in the diffusion barrier.

32. The fermentation apparatus of claim 31 wherein the diffusion barrier comprises a polymer matrix.

33. The fermentation apparatus of 28 wherein the diffusion barrier comprises at least one of swelling bodies and soluble components which act as pore forming agents.

34. The fermentation apparatus of claim 33 wherein the diffusion barrier comprises swelling bodies which increase in volume as a function of at least one of pH and temperature of the culture liquid.

35. The fermentation apparatus of claim 33 wherein the diffusion barrier comprises soluble components which act as pore forming agents dissolve as a function of at least one of pH and temperature of the culture liquid.
